(19) United States
(12) Patent Application Publication
(10) Pub. No.: US 2025/0090255 A1
(43) Pub. Date: Mar. 20, 2025

(54) WIRE ELONGATION COMPENSATION SYSTEM

(71) Applicant: EndoQuest Robotics, Inc., Houston, TX (US)

(72) Inventors: Raymond Lee, Houston, TX (US); Yongman Park, Houston, TX (US); Sungwoo Cho, Richmond, TX (US); Daniel Kim, Houston, TX (US); Dongsuk Shin, Houston, TX (US)

(73) Assignee: EndoQuest Robotics, Inc., Houston, TX (US)

(21) Appl. No.: 18/965,829

(22) Filed: Dec. 2, 2024

Related U.S. Application Data
(63) Continuation of application No. 18/122,029, filed on Mar. 15, 2023.
Application No. 18/122,029 is a continuation of application No. PCT/US2022/051255, filed on Nov. 29, 2022.
(60) Provisional application No. 63/319,841, filed on Mar. 15, 2022.
(60) Provisional application No. 63/284,512, filed on Nov. 30, 2021.

(51) Int. Cl. A61B 34/37; A61B 34/00; A61B 34/30

(52) U.S. Cl. CPC A61B 34/37; A61B 2034/301; A61B 2034/715

(57) ABSTRACT

A tension compensation system for a robotically controlled medical device can include an instrument controller configured to operatively connect to a robotically controlled medical device to provide tension to one or more actuation cables of the robotically controlled medical device. The system can include an instrument control module configured to control the instrument controller to provide a compensation tension to at least one actuation cable of the one or more actuation cables to compensate for elongation of the at least one actuation cable of the one or more actuation cables based on actuation data associated with the at least one actuation cable of the one or more actuation cables.

### CROSS-REFERENCE TO RELATED APPLICATIONS

This application is a continuation of U.S. patent application Ser. No. 18/122,029 filed Mar. 15, 2023, which is a continuation of International Patent Application No. PCT/US2022/051255 filed Nov. 29, 2022, which claims priority to and the benefit of U.S. Provisional Application No. 63/319,841, filed Mar. 15, 2022 and U.S. Provisional Application No. 63/284,512, filed Nov. 30, 2021, the entire contents of each are herein incorporated by reference in their entirety.

### FIELD

This disclosure relates to robotic surgical systems, e.g., for minimally invasive surgery including, but not limited to, endoluminal and single-site surgery.

### BACKGROUND

Minimally invasive surgery such as endoluminal and single-site robotic surgery offer significant advantages versus traditional robotic surgery. For example, in endoluminal robotic surgery, no incision need be made to access difficult to access locations within a patient's natural lumen. This dramatically reduces and/or eliminates recovery time and improves procedural safety. A single-site system reduces incisions to a minimum single-site, which reduces an otherwise larger number of incisions to provide access for certain procedures.

Certain endoluminal and single-site robotic surgical systems have been proposed. Examples of such systems and related components can be found in U.S. Pat. No. 10,881,422, as well as U.S. Patent Application Nos. US 20210322046, US 20210322045, US 20190117247, US 20210275266, US 20210267702, US 20200107898, US 20200397457, US 202000397456, US 20200315645, and US 201962914226, all of the above being incorporated by reference herein in their entirety.

Conventional surgical robotics and systems have generally been considered satisfactory for their intended purpose. However, there is still a need in the art for improved robotic surgical systems, devices, methods, controls, and components, especially those configured for endoluminal and single-site surgery. The present disclosure provides improvements in such areas, for example.

### SUMMARY

In accordance with at least one aspect of this disclosure, a tension compensation system for a robotically controlled medical device can include an instrument controller configured to operatively connect to a robotically controlled medical device to provide tension to one or more actuation cables of the robotically controlled medical device. The system can include an instrument control module configured to control the instrument controller to provide a compensation tension to at least one actuation cable of the one or more actuation cables to compensate for elongation of the at least one actuation cable of the one or more actuation cables based on actuation data associated with the at least one actuation cable of the one or more actuation cables.

The at least one actuation cable of the one or more actuation cables can be each actuation cable. The actuation data can include a number of times each actuation cable has been actuated, respectively. In certain embodiments, the actuation data can include an average tension of each actuation cable, respectively. Any suitable actuation data configured to allow determination of elongation of a respective wire and/or a suitable compensation tension to compensate for elongation is contemplated herein.

In certain embodiments, the system can include the robotically controlled medical device. The robotically controlled medical device can include a hub having a data storage medium and a data interface connected to the data storage medium. The instrument controller can be configured to connect to the data interface when the medical device is installed on the instrument controller.

In certain embodiments, the actuation data can be stored on the robotically controlled medical device. The instrument control module can be configured to read the actuation data from the data storage medium of the robotically controlled medical device to determine a compensation tension of each actuation cable, respectively.

In certain embodiments, the data storage medium can include a unique instrument identification. In certain embodiments, the actuation data can be stored off of the robotically controlled medical device and associated with the unique instrument identification. Any other suitable storage location and/or scheme to associate actuation data with respective actuation wires of a robotically controlled medical device are contemplated herein.

In certain embodiments, the instrument controller can include an independent motor for each actuation wire. In certain embodiments, the actuation data can include an actuation cycle count. In certain embodiments, each time a respective independent motor is cycled, the instrument control module can increment the actuation cycle count for the respective actuation cable.

In certain embodiments, the instrument control module 107 can be configured to pre-compensate a compensation tension to the at least one actuation cable before an actuation cycle begins. In certain embodiments, the system can include a force sensor mounted on the at least one actuation cable (e.g., on each cable) and configured to detect an actual tension of a respective actuation cable. The control module can be configured to automatically calibrate the compensate tension to the actuation cable during the actuation cycle in response to the detected actual tension from the force sensor.

In accordance with at least one aspect of this disclosure, a robotically controlled medical device can include one or more actuation cables, and a hub. The hub can include a data storage medium configured to store actuation data of each of the one or more actuation cables, and/or a unique identification to be correlated to actuation data stored elsewhere. The hub can also include a data interface connected to the data storage medium, the storage medium being configured to connect to an instrument controller when the medical device is installed on the instrument controller for an instrument control module to access data in the data storage medium.

In accordance with at least one aspect of this disclosure, an instrument control module can be configured to control actuation of an instrument controller to control a robotically controlled medical device that has one or more actuation cables. The instrument control module can also be configured to provide a compensation tension to at least one actuation cable of the one or more actuation cables to compensate for elongation of the at least one actuation cable of the one or more actuation cables based on actuation data associated with the at least one actuation cable of the one or more actuation cables. The instrument control module and/or actuation data can be the same or similar to any embodiments disclosed herein, e.g., as described above.

In accordance with at least one aspect of this disclosure, a non-transitory computer readable medium can include computer executable instructions configured to cause a computer to perform a method. The method can include receiving actuation data associated with the at least one actuation cable of one or more actuation cables of a robotically controlled medical device attached to an instrument controller, and actuating one or more motors of the instrument controller to provide a compensation tension to at least one actuation cable of one or more actuation cables to compensate for elongation of the at least one actuation cable of the one or more actuation cables based on actuation data associated with the at least one actuation cable of the one or more actuation cables. The at least one actuation cable of the one or more actuation cables can be each actuation cable, for example. The actuation data can include any suitable actuation data, e.g., as described above. For example, the actuation data can include an actuation cycle count, and each time the one or more motors of the instrument controller are cycled, the instrument control module can increments the actuation cycle count for a respective actuation cable associated with the one or more motors. The method can include any other suitable method(s) and/or portion(s) thereof.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a schematic view of an embodiment of a system in accordance with this disclosure;

FIG. 2 is an elevation view of an embodiment of a robotically controlled medical device in accordance with this disclosure;

FIG. 3 illustrates cross-sectional, perspective, exploded view of an embodiment of a shaft of the embodiment of FIG. 2, showing one or more actuation wires;

FIG. 4 is a perspective view of a hub of the embodiment of FIG. 2;

FIG. 5A is a perspective view of an embodiment of a chip in accordance with this

disclosure;

FIG. 5B is a rear perspective view of the embodiment of FIG. 5A; and

FIG. 5C is a cross-sectional view of an embodiment of the hub of FIG. 4, showing the chip of FIG. 5A mounted therein.

### DETAILED DESCRIPTION

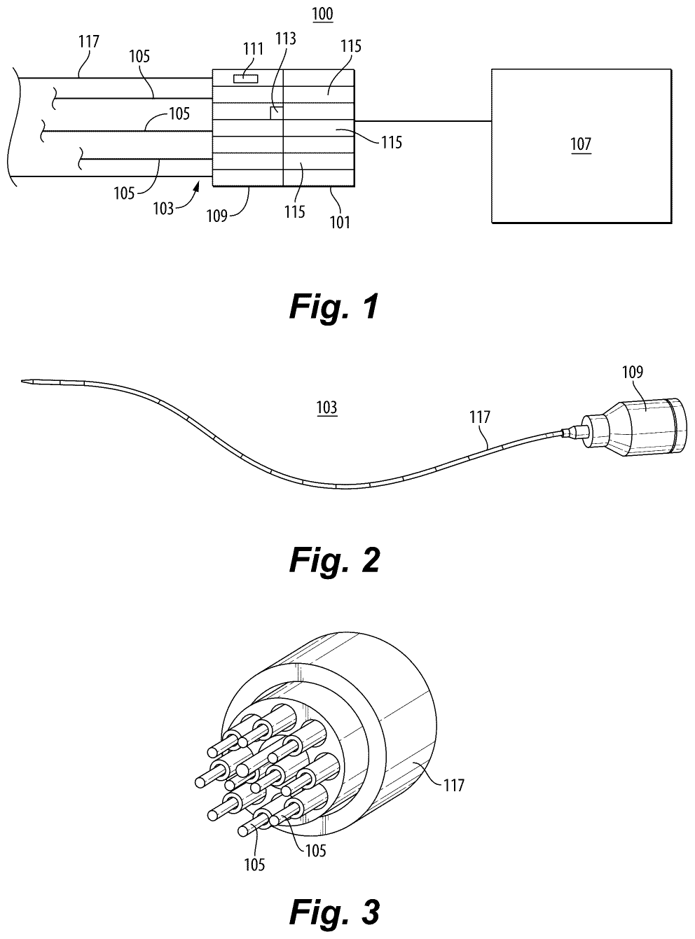

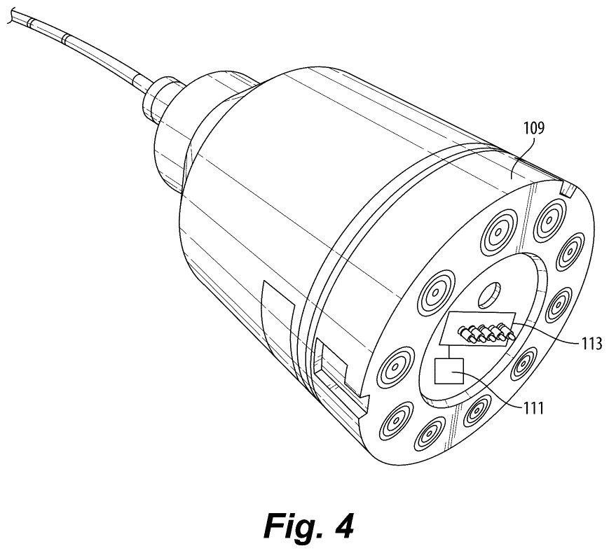

Reference will now be made to the drawings wherein like reference numerals identify similar structural features or aspects of the subject disclosure. For purposes of explanation and illustration, and not limitation, an illustrative view of an embodiment of a system in accordance with the disclosure is shown in FIG. 1 and is designated generally by reference character 100. Other embodiments and/or aspects of this disclosure are shown in FIGS. 2-5C.

FIG. 1 is a schematic view of an embodiment of a system 100 in accordance with this disclosure. Referring to FIGS. 1-4, a tension compensation system 100 for a robotically controlled medical device 103 can include an instrument controller 101 configured to operatively connect to a robotically controlled medical device 103 to provide tension to one or more actuation cables 105 of the robotically controlled medical device 103. The system 100 can include an instrument control module 107 operatively connected to the instrument controller 101. The instrument control module 107 can be configured to control the instrument controller 101 to provide a compensation tension to at least one actuation cable 105 of the one or more actuation cables 105 to compensate for elongation of the at least one actuation cable 105 of the one or more actuation cables 105 based on actuation data associated with the at least one actuation cable 105 of the one or more actuation cables 105.

The at least one actuation cable 105 of the one or more actuation cables 105 can be each actuation cable 105. The actuation data can include a number of times each actuation cable 105 has been actuated, respectively. In certain embodiments, the actuation data can include an average tension of each actuation cable 105, respectively. Any suitable actuation data configured to allow determination of elongation of a respective wire and/or a suitable compensation tension to compensate for elongation is contemplated herein.

The instrument control module 107 can be configured to correlate the actuation data (e.g., a number of tension cycles, and average tension, etc.) to a tension to be applied to account for an elongation of the actuation cables. The data correlating elongation/compensatory tension to the actuation data can be in the form of a lookup table, and can be based on a priori data (e.g., which can be a function of each cable type, material composition, etc.).

In certain embodiments, the system 100 can include the robotically controlled medical device 103. The robotically controlled medical device 103 can include a hub 109 having a data storage medium 111 and a data interface 113 connected to the data storage medium 111. The instrument controller 101 can be configured to connect to the data interface 113 when the medical device 103 is installed on the instrument controller 101.

In certain embodiments, the actuation data can be stored on the robotically controlled medical device 103. The instrument control module 107 can be configured to read the actuation data from the data storage medium 111 of the robotically controlled medical device 103 to determine a compensation tension of each actuation cable 105, respectively.

In certain embodiments, the data storage medium 111 can include a unique instrument identification (e.g., a serial number). In certain embodiments, the actuation data can be stored off of the robotically controlled medical device 103 and associated with the unique instrument identification (e.g., such that all data is stored accessible to the instrument control module 107). For example, the instrument control module 107 can be configured to store the data of each cable 105 of each medical device 103 (e.g., within a desired data age limits) correlated to the respective unique instrument identification, and the instrument control module 107 can store the data after the medical device 103 is disconnected from the instrument controller 101. The instrument control module 107 can then look up actuation data when the medical device 103 is reconnected at some point in the future (e.g., later in the same procedure or for a different patient for reusable devices) to determine a suitable tension compensation. Any suitable storage location and/or scheme to associate actuation data with respective actuation wires of a robotically controlled medical device 103 is contemplated herein.

In certain embodiments, the instrument controller 101 can include an independent motor 115 (e.g., a push motor) for each actuation wire 105. In certain embodiments, the actuation data can include an actuation cycle count. In certain embodiments, each time a respective independent motor 115 is cycled, the instrument control module 107 can increment the actuation cycle count for the respective actuation cable 105. In this regard, the instrument control module 107 can add additional tension (e.g., to increase stroke length by pushing a motor slightly more forward in a push motor arrangement as shown) to a respective cable 105 to account for elongation with each cycle. In some embodiments, the instrument control module 107 can pre-compensate a compensation tension to the actuation cable 105 before the actuation cycle starts. In other embodiments, a force sensor (not shown, such as a load cell) can be further mounted on the actuation cable 105 and configured to detect the tension of the actuation cable 105, and thereby, during the actuation cycle, the instrument control module 107 can automatically calibrate a compensate tension to the actuation cable 105 in response to the detected tension from the force sensor.

In accordance with at least one aspect of this disclosure, a robotically controlled medical device 103 can include one or more actuation cables 105, and a hub 109. The hub 109 can include a data storage medium 111 configured to store actuation data of each of the one or more actuation cables 105, and/or a unique identification to be correlated to actuation data stored elsewhere. The hub 109 can also include a data interface 113 connected to the data storage medium 111. The data storage medium 111 can be configured to connect to an instrument controller 101 when the medical device 103 is installed on the instrument controller 101 for an instrument control module 107 to access data in the data storage medium 111.

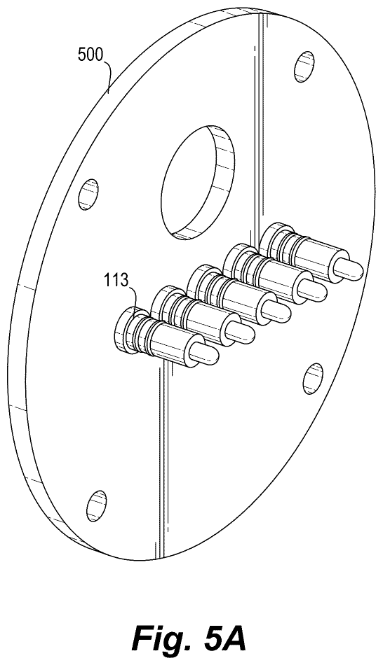

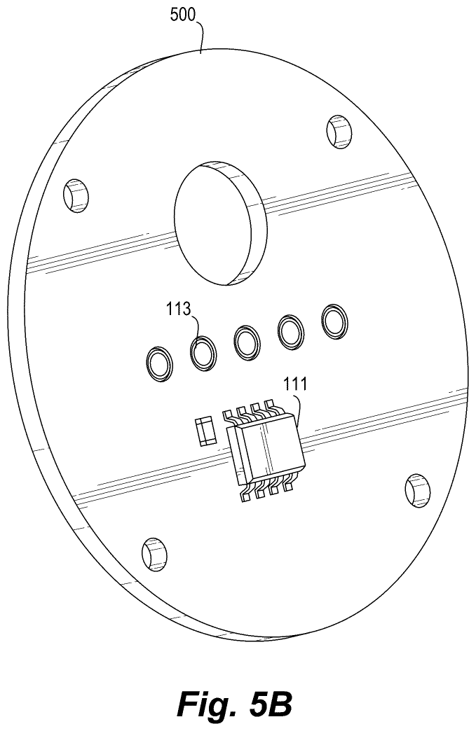

FIG. 2 is an elevation view of an embodiment of a robotically controlled medical device 103 in accordance with this disclosure. FIG. 3 illustrates a cross-sectional, perspective, exploded view of an embodiment of a shaft 117 of a medical device 103, showing one or more actuation cables 105. FIG. 4 is a perspective view of a hub 109 of the medical device 103.

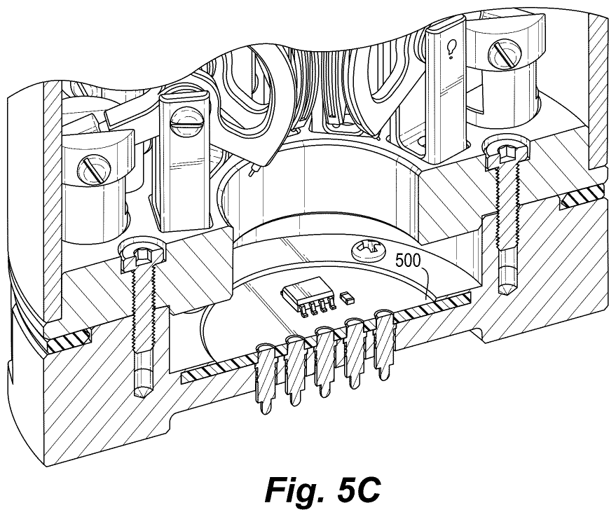

FIG. 5A is a perspective view of an embodiment of a chip 500 hosting the data interface 113 and the storage medium 111. FIG. 5B is a rear perspective view of the embodiment of FIG. 5A. FIG. 5C is a cross-sectional view of an embodiment of the hub of FIG. 4, showing the chip 500 of FIG. 5A mounted therein. For example, the chip 500 can be secured to an inner surface of a proximal housing portion, e.g., via one or more fasteners (e.g., via a screw as shown) and/or with a washer as shown. The proximal housing portion can be secured to a distal housing portion, e.g., via one one or more fasteners (e.g., a plurality of screws as shown).

In accordance with at least one aspect of this disclosure, an instrument control module (e.g., module 107 as described above) can be configured to control actuation of an instrument controller (e.g., controller 101 as described above) to control a robotically controlled medical device (e.g., device 103 as described above) that has one or more actuation cables (e.g., cables 105 as described above). The instrument control module can also be configured to provide a compensation tension to at least one actuation cable of the one or more actuation cables to compensate for elongation of the at least one actuation cable of the one or more actuation cables based on actuation data associated with the at least one actuation cable of the one or more actuation cables. The instrument control module and/or actuation data can be the same or similar to any embodiments disclosed herein, e.g., as described above.

In accordance with at least one aspect of this disclosure, a non-transitory computer readable medium can include computer executable instructions configured to cause a computer to perform a method. The method can include receiving actuation data associated with at least one actuation cable of one or more actuation cables (e.g., cables 105 as described above) of a robotically controlled medical device (e.g., device 103 as described above) attached to an instrument controller (e.g., controller 101 as described above), and actuating one or more motors of the instrument controller to provide a compensation tension to at least one actuation cable of one or more actuation cables to compensate for elongation of the at least one actuation cable of the one or more actuation cables based on actuation data associated with the at least one actuation cable of the one or more actuation cables. The at least one actuation cable of the one or more actuation cables can be each actuation cable, for example. The actuation data can include any suitable actuation data, e.g., as described above. For example, the actuation data can include an actuation cycle count, and each time the one or more motors of the instrument controller are cycled, the instrument control module can increment the actuation cycle count for a respective actuation cable associated with the one or more motors. The method can include any other suitable method(s) and/or portion(s) thereof.

Certain embodiments include a wire elongation compensation system, e.g., for tungsten control wires. Embodiments can enable monitoring and storing activation data per motor/wire and use a lookup table to increase stroke length of motor a based on how many uses a tungsten control wire has over time.

Various actuation components, including tungsten wires, for example, can deform overtime under stress. Performance of the traditional reusable instruments, e.g., in gripping, can be degraded by about 25% over 10 cycles due to elongation of actuation wires and friction in various actuation components.

In certain embodiments, instrument actuation information can be stored on a memory chip built in for example, the data interface 113 of the medical device. Each actuation wire can be controlled by an independent motor 115, and tension of each actuation wire can be adjusted by system software. Actuation information stored on the medical device's memory chip can be utilized to predict tension to be compensated by system software to deliver desired performance.

Embodiments can be utilized with any suitable robotically controlled medical device or system (e.g., a robotic endoluminal surgical system).

Any module(s) disclosed herein can include any suitable hardware and/or software module(s) configured to perform any suitable function(s) (e.g., as disclosed herein, e.g., as described above). As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A tension compensation system for a robotically controlled medical device, comprising:
an instrument controller configured to operatively connect to a robotically controlled medical device to provide tension to one or more actuation cables of the robotically controlled medical device; and
an instrument control module configured to control the instrument controller to provide a compensation tension to at least one actuation cable of the one or more actuation cables to compensate for elongation of the at least one actuation cable of the one or more actuation cables based on actuation data associated with the at least one actuation cable of the one or more actuation cables.

2. The system of claim 1, wherein the at least one actuation cable of the one or more actuation cables is each actuation cable.

3. The system of claim 2, wherein the actuation data includes a number of times each actuation cable has been actuated, respectively.

4. The system of claim 2, wherein the actuation data includes an average tension of each actuation cable, respectively.

5. The system of claim 2, further comprising the robotically controlled medical device.

6. The system of claim 5, wherein the robotically controlled medical device includes a hub having:
a data storage medium; and
a data interface connected to the data storage medium, wherein the instrument controller is configured to connect to the data interface when the medical device is installed on the instrument controller.

7. The system of claim 6, wherein the actuation data is stored on the robotically controlled medical device.

8. The system of claim 7, wherein the instrument control module is configured to read the actuation data from the data storage medium of the robotically controlled medical device to determine a compensation tension of each actuation cable, respectively.

9. The system of claim 6, wherein the data storage medium includes a unique instrument identification, wherein the actuation data is stored off of the robotically controlled medical device and associated with the unique instrument identification.

10. The system of claim 2, wherein the instrument controller includes an independent motor for each actuation wire, wherein the actuation data includes an actuation cycle count, wherein each time a respective independent motor is cycled, the instrument control module increments the actuation cycle count for the respective actuation cable.

11. A robotically controlled medical device, comprising:
one or more actuation cables; and
a hub having:
a data storage medium configured to store actuation data of each of the one or more actuation cables, and/or a unique identification to be correlated to actuation data stored elsewhere; and
a data interface connected to the data storage medium, the storage medium being configured to connect to an instrument controller when the medical device is installed on the instrument controller for an instrument control module to access data in the data storage medium.

12. An instrument control module configured to control actuation of an instrument controller to control a robotically controlled medical device that has one or more actuation cables, wherein the instrument control module is configured to:
provide a compensation tension to at least one actuation cable of the one or more actuation cables to compensate for elongation of the at least one actuation cable of the one or more actuation cables based on actuation data associated with the at least one actuation cable of the one or more actuation cables.

13. The instrument control module of claim 12, wherein the at least one actuation cable of the one or more actuation cables is each actuation cable.

14. The instrument control module of claim 13, wherein the actuation data includes a number of times each actuation cable has been actuated, respectively.

15. The instrument control module of claim 13, wherein the actuation data includes an average tension of each actuation cable, respectively.

16. The instrument control module of claim 13, wherein the instrument control module is configured to read the actuation data from a data storage medium of the robotically controlled medical device to determine a compensation tension of each actuation cable, respectively.

17. The instrument control module of claim 16, wherein the actuation data includes an actuation cycle count, wherein each time a respective independent motor of the instrument controller is cycled, the instrument control module increments the actuation cycle count for the respective actuation cable.
